(19) United States
(12) Patent Application Publication
(10) Pub. No.: US 2026/0345874 A1
(43) Pub. Date: Nov. 5, 2026

(54) MEDICAL DEVICES WITH POLYURETHANE HAVING NATURALLY FORMING HYDRATION LAYER

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US)

(73) Assignee: Boston Scientific Scimed, Inc., Maple Grove, MN (US)

(21) Appl. No.: 19/666,883

(22) Filed: May 4, 2026

Related U.S. Application Data
(60) Provisional application No. 63/800,234, filed on May 5, 2025.

(51) Int. Cl. A61L 31/10; A61M 25/09

(52) U.S. Cl. CPC A61L 31/10; A61M 25/09; A61M 2025/09133

(57) ABSTRACT

In some aspects, the present disclosure provides medical devices that comprise a polyurethane region comprising: (a) an end-capped polyurethane that comprises (i) a linear central portion comprising residues of a linear hydrophilic polymer diol, residues of a diisocyanate, and residues of a C₂-C₁₄ diol and (ii) an end cap on each end of the linear central portion, each end cap comprising a residue of a hydroxyl-terminated hydrophilic polymer that is linked to the central portion through a urethane linkage and/or (b) a comb-shaped polyurethane that comprise residues of a diisocyanate and residues of a hydrophilic-polymer-tailing diol that contains a diol head and at least one hydrophilic polymer tail attached to the diol head.

### CROSS-REFERENCE TO RELATED APPLICATIONS

This application claims the benefit of U.S. Provisional Patent Application Serial No. 63/800,234 filed on May 5, 2025, the disclosure of which is incorporated herein by reference.

### FIELD

The present disclosure relates to medical devices formed with polyurethanes having naturally forming hydration layers and to methods of making the same.

### BACKGROUND

The Sensor® Nitinol Guidewire with Hydrophilic Tip from Boston Scientific (Marlborough MA, USA) is a hybrid guidewire designed for urologic procedures. For example, the Sensor® guidewire is commonly used in urologic procedures, including ureteroscopy, stone management, and other interventions involving the urinary tract. The Sensor® guidewire features a kink-resistant nitinol core, a flat-wire stainless steel construction designed for enhanced instrumentation and device placement, and a PFTE coating designed to provide tactile feedback and a smooth surface to facilitate advancement and device tracking. The Sensor® guidewire further features a 5 cm hydrophilic coated distal end designed to facilitate access past obstructions and passage through tortuous anatomy. With reference to FIG. 1, the distal end of the Sensor® guidewire includes a nitinol core wire 110 that is coated with a co-extruded bilayer containing a Bynel™ linear low-density polyethylene tie layer 120 adjacent the nitinol core, and a Tecothane™ aromatic polyether-based thermoplastic polyurethane layer 130 adjacent the tie layer 120. An outer hydrophilic coating 140 is provided over the bilayer in a multi-step process.

### SUMMARY

In some aspects, the present disclosure provides medical devices that comprise a polyurethane region comprising: (a) an end-capped polyurethane that comprises (i) a linear central portion comprising residues of a linear hydrophilic polymer diol, residues of a diisocyanate, and residues of a C₂-C₁₄ diol and (ii) an end cap on each end of the linear central portion, each end cap comprising a residue of a hydroxyl-terminated hydrophilic polymer that is linked to the central portion through a urethane linkage and/or (b) a comb-shaped polyurethane that comprise residues of a diisocyanate and residues of a hydrophilic-polymer-tailing diol that contains a diol head and at least one hydrophilic polymer tail attached to the diol head.

Some embodiments, the polyurethane region comprises the end-capped polyurethane that comprises (i) a linear central portion comprising residues of a linear hydrophilic polymer diol, residues of a diisocyanate, and residues of a C₂-C₁₄ diol and (ii) an end cap on each end of the linear central portion, each end cap comprising a residue of a hydroxyl-terminated hydrophilic polymer that is linked to the central portion through a urethane linkage. In some of these embodiments, the diisocyanate is an aliphatic or aromatic diisocyanate. In some of these embodiments, the diisocyanate is selected from 4,4′-methylenediphenyl diisocyanate (MDI), 4,4'-methylenedicyclohexyl diisocyanate (H₁₂MDI), and 1,6-hexamethylene diisocyanate (HDI). In some of these embodiments, the linear hydrophilic polymer diol is selected from a poly(ethylene oxide) having terminal hydroxyl groups, a poly(propylene oxide) having terminal hydroxyl groups, a poly(ethylene oxide-co-propylene oxide) having terminal hydroxyl groups, a poly(tetramethylene oxide) having terminal hydroxyl groups, and a polyester having terminal hydroxyl groups. In some of these embodiments, the C₂-C₁₄ diol is selected from C₂-C₁₄ aliphatic diols and C₂-C₁₄ ether diols.

In some embodiments, the polyurethane region comprises the comb-shaped polyurethane that comprise residues of a diisocyanate and residues of a hydrophilic-polymer-tailing diol that contains a diol head and at least one hydrophilic polymer tail attached to the diol head. In some of these embodiments, the hydrophilic-polymer-tailing diol contains between 1 and 20 hydrophilic polymer tails attached to the diol head. In some of these embodiments the at least one hydrophilic polymer tail is selected from a poly(ethylene oxide) tail, a poly(propylene oxide) tail, a poly(ethylene oxide-co-propylene oxide) tail, a poly(tetramethylene oxide) tail, and a polyester tail. In some of these embodiments, the diisocyanate is an aliphatic or aromatic diisocyanate. In some of these embodiments, the diisocyanate is selected from 4,4′-methylenediphenyl diisocyanate (MDI), 4,4'-methylenedicyclohexyl diisocyanate (H₁₂MDI), and 1,6-hexamethylene diisocyanate (HDI). In some of these embodiments, the comb-shaped polyurethane further comprises residues of a linear hydrophilic polymer diol, which, for example, may be selected from a poly(ethylene oxide) having terminal hydroxyl groups, a poly(propylene oxide) having terminal hydroxyl groups, a poly(ethylene oxide-co-propylene oxide) having terminal hydroxyl groups, a poly(tetramethylene oxide) having terminal hydroxyl groups, and a polyester having terminal hydroxyl groups. In some of these embodiments, the comb-shaped polyurethane may further comprise residues of a C₂-C₁₄ diol, which may be selected from C₂-C₁₄ aliphatic diols and C₂-C₁₄ ether diols.

In some embodiments, which can be used in conjunction with the above aspects and embodiments, the polyurethane region is a polyurethane layer disposed over a substrate. In some of these embodiments, the substrate is a metallic substrate. In some of these embodiments, a tie layer is provided between the polyurethane outer layer and the substrate.

In some embodiments, which can be used in conjunction with the above aspects and embodiments, medical devices for use in conjunction with the present disclosure are selected from guidewires, leads, catheters, endoscopes, occlusive devices, artificial heart valves, anchors, stents, ports, pumps, tubing, ventricular assist devices, filters, baskets, meshes, and drug delivery devices, among others.

The medical devices of the present disclosure are advantageous relative to certain prior designs in that device design is simplified through the need for a reduced number of layers. As a result, less processing steps are required to form the devices.

The above and other aspects, embodiments, features and benefits of the present disclosure will be readily apparent from the following detailed description.

### BRIEF DESCRIPTION OF THE DRAWINGS

FIG. 1 schematically illustrates a cross-section of a guidewire, in accordance with the prior art.

FIG. 2 schematically illustrates a cross-section of a guidewire, in accordance with an embodiment of the present disclosure.

FIG. 3 schematically illustrates method of forming an end-capped polyurethane, in accordance with an embodiment of the present disclosure.

FIG. 4 schematically illustrates method of forming a comb-shaped polyurethane, in accordance with an embodiment of the present disclosure.

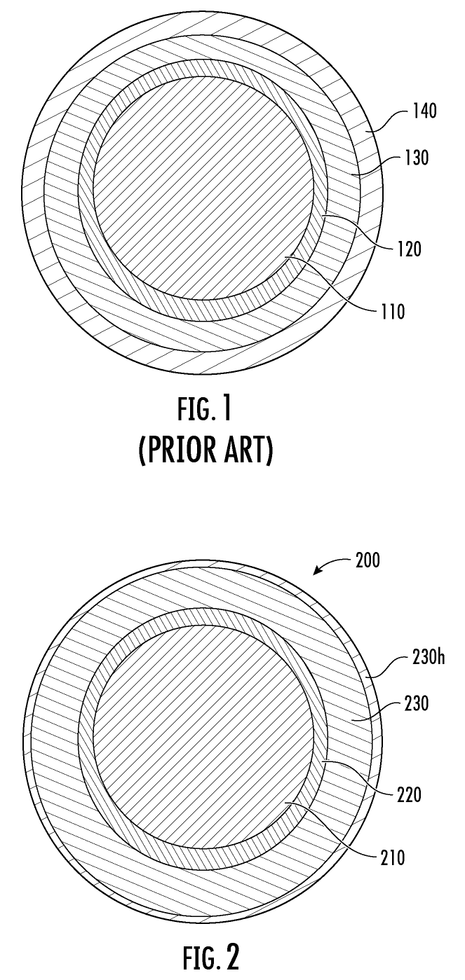

### DETAILED DESCRIPTION

In various aspects, the present disclosure provides polyurethanes that can be used to form polymeric regions for a variety of medical devices. The polymeric regions include polymeric regions that correspond to medical device coatings, polymeric regions that correspond to medical device components, and polymeric regions that correspond to entire medical devices. The polymeric regions may further comprise additional agents as discussed below.

In particular embodiments, and with reference to FIG. 2, the present disclosure provides medical devices 200 that comprise the following: a metallic substrate 210, a tie layer 220 in contact with the metallic substrate 210, and a coating layer 230 in contact with the tie layer 220. The coating layer 230 comprises an end-capped polyurethane and/or a comb-shaped polyurethane as described herein. The end-capped and comb-shaped polyurethanes described herein have hydrophilic polymer end caps and hydrophilic polymer side groups, respectively, that allow a surface hydration layer 230h to be naturally formed at a surface of the coating layer 230 when the coating layer 230 comes into contact with an aqueous fluid, such as a body fluid (e.g., urine, blood, gastric juices, lymph, cerebrospinal fluid, synovial fluid, pleural fluid, pericardial fluid, bile, amniotic fluid, peritoneal fluid, etc.), normal saline, phosphate buffered saline and water, among numerous other aqueous fluids.

Examples of metals for forming metallic substrates for the devices of the present disclosure include metals, such as titanium, tungsten, gold, platinum, iridium, tantalum, , silver, copper, and aluminum, and metal alloys, such as stainless steel, nickel-titanium alloys (e.g., nitinol), and cobalt-chromium alloys such as Elgiloy® (a cobalt-chromium-nickel-molybdenum alloy). The metallic substrates may be in various shapes including cylindrical, plate-shaped, spherical and spinal-shaped, among others.

Examples of materials for tie layer include silicone, polyamide-polyether block copolymers (e.g., PEBAX® and VESTAMID® E), maleic anhydride grafted polyolefins including maleic anhydride grafted polyolefins, such as Bynel®, Admer® and Orevac®, ethylene vinyl acetate copolymers, ethylene methyl acrylate copolymers, and ethylene acrylic acid copolymers, among others.

Polyurethanes for use in the present disclosure include end-capped polyurethanes that comprise (a) a linear central portion comprising residues of a linear hydrophilic polymer diol, residues of a diisocyanate, and residues of a C₂-C₁₄ diol and (b) an end cap on each end of the linear central portion. Each end cap comprises a residue of a hydroxyl-terminated hydrophilic polymer that is linked to the central portion through a urethane linkage. In various embodiments, the end-capped polyurethanes are thermoplastic end-capped polyurethanes.

Examples of hydrophilic polymer diol residues for use in the polyurethanes of the present disclosure may be selected, for example, from residues of the following polymer diols: polyether diols including poly(C₁-C₆-alkylene oxide) diols such as poly(ethylene oxide) diols (also referred to as polyethylene glycol diols), poly(propylene oxide) diols, poly(ethylene oxide-co-propylene oxide) diols, poly(tetramethylene oxide) diols, poly(hexamethylene oxide) diols, polyester diols including polyglycolide diols, polylactide diols, poly(lactide-co-glycolide) diols, poly(β-propiolactone) diols, poly(β-butyrolactone) diols, poly(γ-butyrolactone) diols, poly(γ-valerolactone) diols, poly(δ-valerolactone) diols, and poly(ε-caprolactone) diols, polyoxazoline diols including poly(2-C₁-C₆-alkyl-2-oxazoline diols) such as poly(2-methyl-2-oxazoline) diols, poly(2-ethyl-2-oxazoline) diols, poly(2-propyl-2-oxazoline) diols, poly(2-isopropyl-2-oxazoline) diols, and poly(2-n-butyl-2-oxazoline) diols, poly(2-phenyl-2-oxazoline) diols, polymer diols formed from one or more polar aprotic vinyl monomers, including poly(N-vinyl pyrrolidone) diols, poly(acrylamide) diols, poly(N-methyl acrylamide) diols, poly(dimethyl acrylamide) diols, poly(N-vinylimidazole) diols, poly(4-vinylimidazole) diols, and poly(sodium 4-vinylbenzenesulfonate) diols, polydioxanone diols, and poly(N-isopropylacrylamide) diols, among others.

Hydrophilic polymer diol residues in the polyurethanes of the present disclosure typically contain between 5 and 10000 monomer units or more, for example ranging anywhere from 5 to 10 to 25 to 50 to 100 to 250 to 500 to 1000 to 250 to 50000 to 10000 monomer units (in other words, ranging between any two of the preceding values).

In some embodiments, the hydrophilic polymer diol residues may constitute about 1 to about 70 weight percent of the end-capped polyurethanes, for example ranging anywhere from 1 to 2 to 5 to 10 to 25 to 50 to 70 weight percent of the end-capped polyurethanes.

Examples of diisocyanate residues for use in the polyurethanes of the present disclosure include residues of aromatic diisocyanates and residues of aliphatic diisocyanates, among others. Particular examples include residues of the following: 4,4′-methylenediphenyl diisocyanate (MDI), 4,4′-diisocyanato dicyclohexylmethane (also known as 4,4'-methylenedicyclohexyl diisocyanate, hydrogenated MDI or H₁₂MDI), 1,4-tetramethylene diisocyanate, 1,6-hexamethylene diisocyanate (HDI), toluene diisocyanate (TDI), 1,5-naphthalene diisocyanate, para-phenylene diisocyanate, 3,3′-tolidene-4,4′diisocyanate, 3,3′-dimethyl-diphenylmethane-4,4′-diisocyanate, 1-isocyanato-3-isocyanatomethyl-3,5,5-trimethyl-cyclohexane (isophorone diisocyanate), and combinations thereof.

In some embodiments, the diisocyanate residues may comprise from about 1 to about 70 weight percent of the end-capped polyurethanes, for example ranging anywhere from 1 to 2 to 5 to 10 to 25 to 50 to 70 weight percent of the end-capped polyurethanes.

Examples of C₂-C₁₄ diol residues for use in the polyurethanes of the present disclosure include residues of C₂-C₁₄ aliphatic diols and residues or C₂-C₁₄ ether diols, among others. Particular examples of C₂-C₁₄ diols include alpha,omega-C₂-C₁₄ linear aliphatic diols, such as 1,2-ethane diol, 1,3-n-propane diol, 1,4-n-butane diol, 1,5-n-pentane diol, 1,6-n-hexane diol, 1,7-n-heptane diol, 1,8-n-octane diol, 1,9-n-nonane diol, 1,10-n-decane diol, and 1,12-n-decane diol, C₂-C₁₄ ether diols, such as diethylene glycol, triethylene glycol, tetraethylene glycol, pentaethylene glycol, hexaethylene glycol, heptaethylene glycol, dipropylene glycol, tripropylene glycol, tetrapropylene glycol, and further C₂-C₁₄ diols such as 1,3-butanediol, neopentylglycol, 1,4-cyclohexanedimethanol (CHDM), and combinations of any of the foregoing.

In some embodiments, the C₂-C₁₄ diol residues may comprise from about 1 to about 10 weight percent of the end-capped polyurethanes, for example ranging anywhere from 1 to 2 to 3 to 4 to 6 to 8 to 10 weight percent of the end-capped polyurethanes

Examples of hydroxyl-terminated hydrophilic polymer residues for use in forming end caps of the polyurethanes of the present disclosure may be selected, for example, from residues of the following hydroxyl-terminated hydrophilic polymers: hydroxyl-terminated polyethers including hydroxyl-terminated poly(C₁-C₆-alkylene oxides) such as hydroxyl-terminated poly(ethylene oxides), hydroxyl-terminated poly(propylene oxides), hydroxyl-terminated poly(ethylene oxide-co-propylene oxides), hydroxyl-terminated poly(tetramethylene oxides),hydroxyl-terminated poly(hexamethylene oxides), hydroxyl-terminated polyesters including hydroxyl-terminated polyglycolides, hydroxyl-terminated polylactides, hydroxyl-terminated poly(lactide-co-glycolides), hydroxyl-terminated poly(β-propiolactones), hydroxyl-terminated poly(β-butyrolactones), hydroxyl-terminated poly(γ-butyrolactones), hydroxyl-terminated poly(γ-valerolactones), hydroxyl-terminated poly(δ-valerolactones), and hydroxyl-terminated poly(ε-caprolactones), hydroxyl-terminated polyoxazolines including hydroxyl-terminated poly(2-C₁-C₆-alkyl-2-oxazolines) such as hydroxyl-terminated poly(2-methyl-2-oxazolines), hydroxyl-terminated poly(2-ethyl-2-oxazolines), hydroxyl-terminated poly(2-propyl-2-oxazolines), hydroxyl-terminated poly(2-isopropyl-2-oxazolines), and hydroxyl-terminated poly(2-n-butyl-2-oxazolines), hydroxyl-terminated poly(2-phenyl-2-oxazolines), hydroxyl-terminated polymers formed from one or more polar aprotic vinyl monomers, including hydroxyl-terminated poly(N-vinyl pyrrolidones), hydroxyl-terminated poly(acrylamides), hydroxyl-terminated poly(N-methyl acrylamides), hydroxyl-terminated poly(dimethyl acrylamides), hydroxyl-terminated poly(N-vinylimidazole), hydroxyl-terminated poly(4-vinylimidazole), and hydroxyl-terminated poly(sodium 4-vinylbenzenesulfonates), hydroxyl-terminated polydioxanones, and hydroxyl-terminated poly(N-isopropylacrylamides), among others.

Hydroxyl-terminated hydrophilic polymer residues for use in the polyurethanes of the present disclosure typically contain between 5 and 5000 monomer units or more, for example ranging anywhere from 5 to 10 to 25 to 50 to 100 to 250 to 500 to 1000 to 2500 to 5000 monomer units.

In some embodiments, the hydroxyl-terminated hydrophilic polymer residues may comprise from about 1 to about 70 weight percent of the end-capped polyurethanes, for example ranging anywhere from 1 to 2 to 5 to 10 to 25 to 50 to 70 weight percent of the end-capped polyurethanes.

The monomer composition of the hydrophilic polymer diol residues in the central portion of the polyurethane may be the same as or different from the monomer composition of the hydroxyl-terminated hydrophilic polymer residues that form the end caps of the polyurethane.

In some embodiments, end-capped polyurethanes in accordance with the present disclosure can be formed by a method that comprises (a) reacting a hydrophilic polymer diol, a C₂-C₁₄ diol, and a stochiometric excess of a diisocyanate to form a prepolymer that comprises residues of the hydrophilic polymer diol, residues of the C₂-C₁₄ diol and residues of the diisocyanate, wherein both ends of the prepolymer are terminated with isocyanate groups and (b) reacting the prepolymer with a hydroxyl-terminated hydrophilic polymer to form end caps, wherein each end cap comprises a residue of the hydroxyl-terminated hydrophilic polymer that is linked to a central portion of the polymer through a urethane linkage.

The polyurethane-forming reactions described herein typically can be carried out at ambient temperature with a suitable catalyst, such as a tin-based catalyst (e.g., stannous octoate). However, the reaction rate can be fostered at elevated temperature; for example, a temperature ranging from 30 to 180 °C, in the presence of a suitable catalyst, such as a tin-based catalyst.

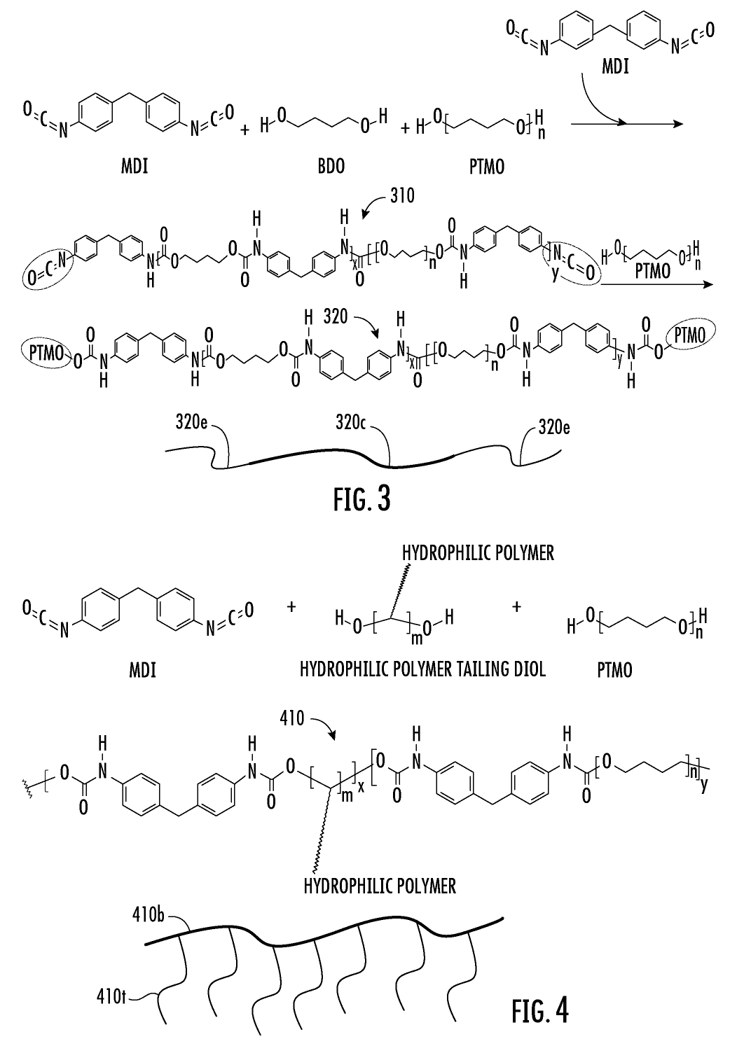

In one particular example and with reference to FIG. 3, a hydrophilic polymer diol, specifically, a poly(tetramethylene oxide) diol (PTMO) where n ranges from 10 to 5000, a C₂-C₁₄ diol, specifically, 1,4-butane diol (BDO), and a stochiometric excess of a diisocyanate, specifically, 4,4′-methylenediphenyl diisocyanate (MDI), are reacted to form an isocyanate-terminated prepolymer 310 that comprises PTMO residues, BDO residues and MDI residues, where x ranges from 5 to 95 and y ranges from 95 to 5. In FIG. 3, MDI is added after the reaction to between the PTMO, BDO and MDI is complete to ensure that essentially all molecules of the isocyanate-terminated prepolymer 310 have terminal isocyanate groups. In a further step, the isocyanate-terminated prepolymer 310 is reacted with a hydroxyl-terminated hydrophilic polymer, specifically, a poly(tetramethylene oxide) diol (PTMO) where n ranges from 10 to 5000, to form an end-capped polyurethane 320 that comprises a central portion (represented schematically by 320c) that is formed from PTMO residues, BDO residues and MDI residues and poly(tetramethylene oxide) end caps (represented schematically by 320e) at each end. Each poly(tetramethylene oxide) end cap comprises a PTMO residue that is linked to a remainder of the polymer through a urethane linkage. In FIG. 3, a large molar excess of PTMO is added to ensure that the PTMO does not form linkages between multiple prepolymers 310. In other embodiments, a poly(tetramethylene oxide) molecule may be employed that has only a single hydroxyl end group.

In some embodiments, end-capped polyurethanes in accordance with the present disclosure can be formed by a method that comprises (a) reacting a hydrophilic polymer diol with a stochiometric excess of a diisocyanate to form a first prepolymer that comprises residues of the hydrophilic polymer diol and residues of the diisocyanate, wherein both ends of the prepolymer are terminated with isocyanate groups, (b) reacting a stochiometric excess of the first prepolymer with a C₂-C₁₄ diol to form a second prepolymer that contains residues of the C₂-C₁₄ diol and residues of the first prepolymer (which in turn contains residues of the hydrophilic polymer diol and residues of the diisocyanate), wherein both ends of the second prepolymer are terminated with isocyanate groups, and (c) reacting the second prepolymer with a hydroxyl-terminated hydrophilic polymer to form end caps, wherein each end cap comprises a residue of the hydroxyl-terminated hydrophilic polymer that is linked to a central portion of the polymer through a urethane linkage.

Polyurethanes for use in the present disclosure also include comb-shaped polyurethanes that comprise residues of a diisocyanate and residues of a hydrophilic-polymer-tailing diol which contains (a) a diol head and (b) at least one hydrophilic polymer tail attached to the diol head. In various embodiments, the comb-shaped polyurethanes are thermoplastic comb-shaped polyurethanes.

Diisocyanate residues suitable for forming the comb-shaped polyurethanes include the diisocyanate residues listed above. In some embodiments, the residues of the diisocyanates may constitute about 5 to about 95 weight percent of the comb-shaped polyurethanes, for example ranging anywhere from 5 to 10 to 15 to 25 to 50 to 75 to 85 to 90 to 95 weight percent of the comb-shaped polyurethanes.

In some embodiments, the hydrophilic-polymer-tailing diol contains between 1 and 20 hydrophilic polymer tails attached to the diol head, for example, the hydrophilic-polymer-tailing diol may contain anywhere from 1 to 2 to 3 to 4 to 5 to 6 to 8 to 10 to 15 to 20 hydrophilic polymer tails attached to the diol head.

Examples of diol heads for the hydrophilic-polymer-tailing diols of the present disclosure include C₃-C₂₀ aliphatic diol heads, C₃-C₂₀ ether diol heads, and other C₃-C₂₀ diol heads.

Examples of hydrophilic polymer tails for the hydrophilic-polymer-tailing diols of the present disclosure may be selected, for example, from the following polymer tails: polyether tails including poly(C₁-C₆-alkylene oxide) tails such as poly(ethylene oxide) (PEO) (also referred to as polyethylene glycol or PEG) tails, poly(propylene oxide) tails, poly(ethylene oxide-co-propylene oxide) tails, poly(tetramethylene oxide) tails, poly(hexamethylene oxide) tails, polyester tails including polyglycolide tails, polylactide tails, poly(lactide-co-glycolide) tails, poly(β-propiolactone) tails, poly(β-butyrolactone) tails, poly(γ-butyrolactone) tails, poly(γ-valerolactone) tails, poly(δ-valerolactone) tails, and poly(ε-caprolactone) tails, polyoxazoline tails including poly(2-C₁-C₆-alkyl-2-oxazoline tails) such as poly(2-methyl-2-oxazoline) tails, poly(2-ethyl-2-oxazoline) tails, poly(2-propyl-2-oxazoline) tails, poly(2-isopropyl-2-oxazoline) tails, and poly(2-n-butyl-2-oxazoline) tails, poly(2-phenyl-2-oxazoline) tails, polymer tails formed from one or more polar aprotic vinyl monomers, including poly(N-vinyl pyrrolidone) tails, poly(acrylamide) tails, poly(N-methyl acrylamide) tails, poly(dimethyl acrylamide) tails, poly(N-vinylimidazole) tails, poly(4-vinylimidazole) tails, and poly(sodium 4-vinylbenzenesulfonate) tails, polydioxanone tails and poly(N-isopropylacrylamide) tails, among others. To avoid crosslinking, the hydrophilic polymer tails to not contain any hydroxyl groups. Hydrophilic polymer tails for the hydrophilic-polymer-tailing diols the present disclosure typically contain between 5 and 5000 monomer units or more, for example ranging anywhere from 5 to 10 to 25 to 50 to 100 to 250 to 500 to 1000 to 2500 to 5000 monomer units.

In some embodiments, residues of the hydrophilic-polymer-tailing diols may constitute about 5 to about 95 weight percent of the comb-shaped polyurethanes, for example ranging anywhere from 5 to 10 to 15 to 25 to 50 to 75 to 85 to 90 to 95 weight percent of the comb-shaped polyurethanes.

In some embodiments, the comb-shaped polyurethanes may further comprise residues of one or more additional diols such as (a) residues of a C₂-C₁₄ diol, examples of which are provided hereinabove and/or (b) residues of a hydrophilic polymer diol, examples of which are provided hereinabove. Where a hydrophilic polymer diol residue is included in the comb-shaped polyurethanes, the monomer composition of the hydrophilic polymer diol residues may be the same as or different from the monomer composition of the hydrophilic-polymer-tailing diol residues.

In some embodiments, the residues of the C₂-C₁₄ diol may constitute about 1 to about 25 weight percent of the comb-shaped polyurethanes, for example ranging anywhere from 1 to 2 to 5 to 10 to 15 to 20 to 25weight percent of the comb-shaped polyurethanes

In some embodiments, the residues of the hydrophilic polymer diol may constitute about 5 to about 95 weight percent of the comb-shaped polyurethanes, for example ranging anywhere from 5 to 10 to 15 to 25 to 50 to 75 to 85 to 90 to 95 weight percent of the comb-shaped polyurethanes.

Comb-shaped polyurethanes in accordance with the present disclosure may be formed by a method that comprises reacting a hydrophilic-polymer-tailing diol with a diisocyanate. In some embodiments, a C₂-C₁₄ diol and/or a hydrophilic polymer diol s present during the reaction.

In one particular example and with reference to FIG. 4, a hydrophilic-polymer-tailing diol, specifically a hydrophilic-polymer-tailing diol having an aliphatic C₃₋₂₀ diol head and containing m hydrophilic polymer tails, where m is an integer ranging from 1 to 20, a hydrophilic polymer diol, specifically, a poly(tetramethylene oxide) diol (PTMO), where n ranges from 10 to 5000, and a diisocyanate, specifically, 4,4′-methylenediphenyl diisocyanate (MDI), are reacted to form a comb-shaped polyurethane 410 that comprises hydrophilic-polymer-tailing diol, PTMO residues, and MDI residues, where x ranges from 5 to 95 and y ranges from 95 to 5. The comb copolymer comprises a backbone (represented schematically by 410b) that is formed from the MDI, the diol head of the hydrophilic-polymer-tailing diol, and the PTMO and “teeth” (represented schematically by 410t) that are formed from the hydrophilic polymer tail(s) of the hydrophilic-polymer-tailing diol.

As previously indicated, in some aspects, the present disclosure provides medical devices that have one or more polymeric regions that comprise the end-capped and/or comb-shaped polyurethanes described herein. The polymeric regions include polymeric regions that correspond to medical device coatings, polymeric regions that correspond to medical device components, and polymeric regions that correspond to entire medical devices.

The polymeric regions may further comprise additional agents. Additional agents for use in the polymeric regions described herein include therapeutic agents, imaging agents, and colorants, among others.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Medical devices for use herein include guidewires, leads, catheters, such as balloon catheters, guide catheters, drug delivery catheters, endoscopes, occlusive devices such as Watchman® (mesh over nitinol) and embolism coils, artificial heart valves, anchors, stents, ports, pumps, tubing, ventricular assist devices, filters, baskets, meshes (from forming fiber into mesh), and drug delivery devices, among others.

With reference again to FIG. 2, in some embodiments, the medical device 200 of FIG. 2 may be a guidewire that includes a nitinol core wire 210, a maleic anhydride grafted polyolefin tie layer 220 in contact with the nitinol core wire 210, and a polyurethane coating layer 230 in contact with the maleic anhydride grafted polyolefin tie layer 220. The polyurethane coating layer 230 be formed from an end-capped polyurethane like that described in FIG. 3 or a comb-shaped polyurethane like that of FIG. 4.

The device 200 may be formed, for example, by dual-layer co-extrusion of the maleic anhydride grafted polyolefin inner tie layer 220 and the polyurethane outer coating layer 230 onto the nitinol core wire 210. This process may involve using a first extruder that contains the maleic anhydride grafted polyolefin and a second extruder that contains the polyurethane. The first and second extruders simultaneously melt and deliver the maleic anhydride grafted polyolefin and the polyurethane to a single extrusion die (e.g., a crosshead die). In the die, the maleic anhydride grafted polyolefin and the polyurethane are joined, shaped and deposited in a coaxial dual layer (which corresponds to the inner tie layer 220 and the outer coating layer 230) onto the nitinol core wire 210 in a single process step.

1. A medical device comprising a polyurethane region that comprises an end-capped polyurethane that comprises (i) a linear central portion comprising residues of a linear hydrophilic polymer diol, residues of a diisocyanate, and residues of a C₂-C₁₄ diol and (ii) an end cap on each end of the linear central portion, each end cap comprising a residue of a hydroxyl-terminated hydrophilic polymer that is linked to the central portion through a urethane linkage.

2. The medical device of claim 1, wherein the diisocyanate is an aliphatic or aromatic diisocyanate.

3. The medical device of claim 1, wherein the diisocyanate is selected from 4,4′-methylenediphenyl diisocyanate (MDI), 4,4'-methylenedicyclohexyl diisocyanate (H₁₂MDI), and 1,6-hexamethylene diisocyanate (HDI).

4. The medical device of claim 1, wherein the linear hydrophilic polymer diol is selected from a poly(ethylene oxide) having terminal hydroxyl groups, a poly(propylene oxide) having terminal hydroxyl groups, a poly(ethylene oxide-co-propylene oxide) having terminal hydroxyl groups, a poly(tetramethylene oxide) having terminal hydroxyl groups, and a polyester having terminal hydroxyl groups.

5. The medical device of claim 1, wherein the C₂-C₁₄ diol is selected from C₂-C₁₄ aliphatic diols and C₂-C₁₄ ether diols.

6. The medical device of claim 1, wherein the C₂-C₁₄ diol is selected from 1,4-butane diol and diethylene glycol.

7. The medical device of claim 1, wherein the polyurethane region is a polyurethane layer that is disposed over a substrate.

8. The medical device of claim 7, wherein the substrate is a metallic substrate.

9. The medical device of claim 7, further comprising a tie layer between the polyurethane outer layer and the substrate.

10. A medical device comprising a polyurethane region that comprises a comb-shaped polyurethane that comprise residues of a diisocyanate and residues of a hydrophilic-polymer-tailing diol that contains a diol head and at least one hydrophilic polymer tail attached to the diol head.

11. The medical device of claim 10, wherein the hydrophilic-polymer-tailing diol contains between 1 and 20 hydrophilic polymer tails attached to the diol head.

12. The medical device of claim 10, wherein the at least one hydrophilic polymer tail is selected from a poly(ethylene oxide) tail, a poly(propylene oxide) tail, a poly(ethylene oxide-co-propylene oxide) tail, a poly(tetramethylene oxide) tail, and a polyester tail.

13. The medical device of claim 10, wherein the diisocyanate is an aliphatic or aromatic diisocyanate.

14. The medical device of claim 10, wherein the diisocyanate is selected from 4,4′-methylenediphenyl diisocyanate (MDI), 4,4'-methylenedicyclohexyl diisocyanate (H₁₂MDI), and 1,6-hexamethylene diisocyanate (HDI).

15. The medical device of claim 10, wherein the comb-shaped polyurethane further comprises residues of a C₂-C₁₄ diol.

16. The medical device of claim 15, wherein the C₂-C₁₄ diol is selected from C₂-C₁₄ aliphatic diols and C₂-C₁₄ ether diols.

17. The medical device of claim 15, wherein the C₂-C₁₄ diol is selected from 1,4-butane diol and diethylene glycol.

18. The medical device of claim 10, wherein the comb-shaped polyurethane further comprises residues of a linear hydrophilic polymer diol.

19. The medical device of claim 10, wherein the polyurethane region is a polyurethane layer that is disposed over a substrate.

20. The medical device of claim 19, further comprising a tie layer between the polyurethane outer layer and the substrate.
